(19) United States
(12) Patent Application Publication
(10) Pub. No.: US 2012/0130341 A1
(43) Pub. Date: May 24, 2012

(54) COMBINED IV BAG AND PUMP SYSTEM AND METHOD

(75) Inventor: Kenneth Whitley, Youngsville, NC (US)

(73) Assignee: CareFusion 303, Inc., San Diego, CA (US)

(21) Appl. No.: 12/950,820

(22) Filed: Nov. 19, 2010

(51) Int. Cl. A61M 5/142

(52) U.S. Cl. 604/506; 604/151

(57) ABSTRACT

An intravenous fluid container is disclosed, wherein the fluid container comprises a bag, a fluid reservoir formed within the bag, an outlet, and a pumping element formed within the bag and fluidically coupled between the fluid reservoir and the outlet.

### BACKGROUND

1. Field

The present disclosure generally relates to administration of medical fluid by infusion and, in particular, relates to systems and methods of providing and pumping medical fluid.

2. Background

Infusion pumps have become commonplace within the healthcare world as a way to precisely administer intravenous (IV) fluids. Use of a pump in place of an elevated fluid container with a simple roller clamp to control the flow of the IV fluid allows more accurate and consistent control of the rate of delivery of the fluid to the patient.

The assembly of tubing, valves, fittings, and needles that connect the fluid container to the patient may be referred to as an “IV set.” IV sets are typically disposable to reduce the risk of infection and contamination. When used with an infusion pump, the IV set includes a segment intended to be manipulated by the pump to cause the fluid to flow at a controlled rate. For a peristaltic type of pump, this may be as simple as a length of tubing that is fitted into the pumping compartment. A typical IV pump system is shown in FIG. 1.

IV sets designed for use with piston-type infusion pumps may have a pumping segment or chamber incorporated into the set, wherein the pumping segment fits into a compartment in the infusion pump as shown in FIG. 2. This configuration provides a sterile and disposable system for administering medical fluids as well as the potential for continuous flow but still has the container of medical fluid hanging above the pump at a height that makes the system unstable and awkward to transport.

### SUMMARY

The IV pump system disclosed herein provides a lower total cost of the disposable elements used to administer medical fluids. In addition, the size and weight of the IV pump system may be reduced and the height of the fluid container reduced to improve the portability and stability of the system.

Certain exemplary embodiments of the present disclosure include an IV fluid container comprising a bag having a fluid reservoir formed within the bag, an outlet, and a pumping element formed within the bag and fluidically coupled between the fluid reservoir and the outlet.

In another embodiment, an IV pump is disclosed. The IV pump comprises a housing, an attachment feature attached to the housing, and an actuation feature attached to the housing. The attachment feature is configured to removably attach to an IV fluid container comprising a bag having a fluid reservoir and a pumping element formed within the bag and an outlet. The pumping element is fluidically coupled between the fluid reservoir and the outlet. The actuation feature is configured to manipulate the pumping element to cause fluid to flow from the fluid reservoir to the outlet.

Certain exemplary embodiments of the present disclosure include an IV fluid container comprising a bag, a fluid reservoir formed within the bag, an outlet, and a pumping element formed within the bag and fluidically coupled between the fluid reservoir and the outlet.

In other embodiments, an IV pump is disclosed that comprises a housing and an attachment feature that is attached to the housing and configured to removably attach to an IV fluid container that includes a bag, a fluid reservoir formed within the bag, an outlet, and a pumping element formed within the bag and fluidically coupled between the fluid reservoir and the outlet. The IV pump also includes an actuation feature that is attached to the housing and configured to manipulate the pumping element to cause fluid to flow from the fluid reservoir to the outlet.

In other embodiments, an IV pumping system is disclosed that includes an IV fluid container comprising a body, a fluid reservoir formed within the body, a outlet attached to the body, and a pumping element formed within the body and fluidically coupled between the fluid reservoir and the outlet, and an IV pump configured to removably attach to the IV fluid container and manipulate the pumping element to cause fluid to flow from the fluid reservoir to the outlet.

In other embodiments, a method is disclosed that provides a medical fluid to a patient. The method includes the steps of attaching an IV set to the patient and to an IV fluid container having a reservoir, a outlet, and an integral pumping element fluidically coupled between the reservoir and the outlet, the reservoir being at least partially filled with the medical fluid, attaching an IV pump to the IV fluid container, the IV pump configured to manipulate the pumping element to cause the medical fluid to flow from the reservoir to the outlet; and activating the IV pump.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a diagram of the prior art method of administering medical fluid to a patient using an IV pump.

FIG. 2 illustrates a pumping segment of a prior art IV set fitting into the pump module of the IV pump of FIG. 1.

FIGS. 3A-3B respectively depict front and bottom views of a fluid container that includes a pumping element according to certain aspects of the present disclosure.

FIG. 4 depicts the fluid container of FIG. 3A with an example IV pumping module according to certain aspects of the present disclosure.

FIG. 5A depicts an embodiment of an IV pump configured to operate in conjunction with the fluid container of FIG. 3A according to certain aspects of the present disclosure.

FIGS. 5B-5C respectively depict front and side views of an embodiment of a fluid container configured to operate with an attached pump according to certain aspects of the present disclosure.

FIG. 5D illustrates a portion of an example internal pumping mechanism of the pump of FIG. 5A according to certain aspects of the present disclosure.

FIG. 5E illustrates another embodiment of a pump configured to operate in conjunction with the fluid container of FIG. 5B according to certain aspects of the present disclosure.

FIGS. 6A-6B illustrate an example operational sequence of the pumping mechanism of FIG. 5C.

FIG. 7A depicts a prior art IV pump system.

FIGS. 7B-7C depict IV pump systems incorporating various embodiments of fluid containers and IV pumps according to certain aspects of the present disclosure.

FIG. 8 is a flowchart illustrating the administration of a medical fluid using a fluid container having an integral pumping chamber according to certain aspects of the present disclosure.

### DETAILED DESCRIPTION

IV pumps are frequently configured to accept a portion of the IV set and to provide the pumping action through manipulation of flexible elements of the IV set. This flexible element may simply be a portion of the tubing of the IV set or may be a flexible pumping element. Fabrication of an IV set having an integral pumping element involves a number of steps to form the pumping element and then form a leak-tight bond between the pumping element and two pieces of tubing. This fabrication adds a significant cost to the IV set compared to the cost of an IV set without an integral pumping element.

The present disclosure provides an overall cost saving by integrating a pumping element into the fluid container. As the fluid container is formed using material and processes similar to or, in some cases, identical to the materials and processes used to form a pumping element, the forming operations can be performed together using less overall material. This presents significant cost advantages as well as other benefits in safety and portability that will be discussed below.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

While the following discussion is directed to the administration of medical fluid to a patient by a nurse using an IV pump, the disclosed methods and configurations may be used for other medical and non-medical applications and may be used by other individuals, for example a patient self-administering a medical fluid at home.

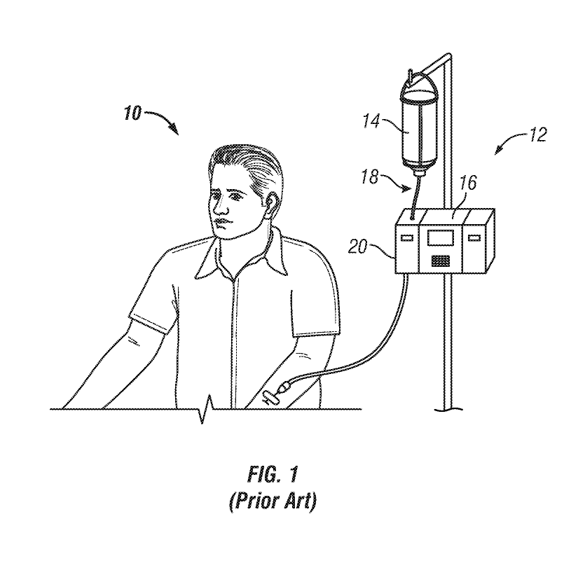

FIG. 1 is a diagram of a method of administering medical fluid to a patient 10 using an IV pump 12. The fluid container 14 is hung at or above the patient's head and connected via an IV set 18 to an IV pump 12 and then to the patient 10. In this example, the IV pump 12 includes a control module 16 and a pumping module 20.

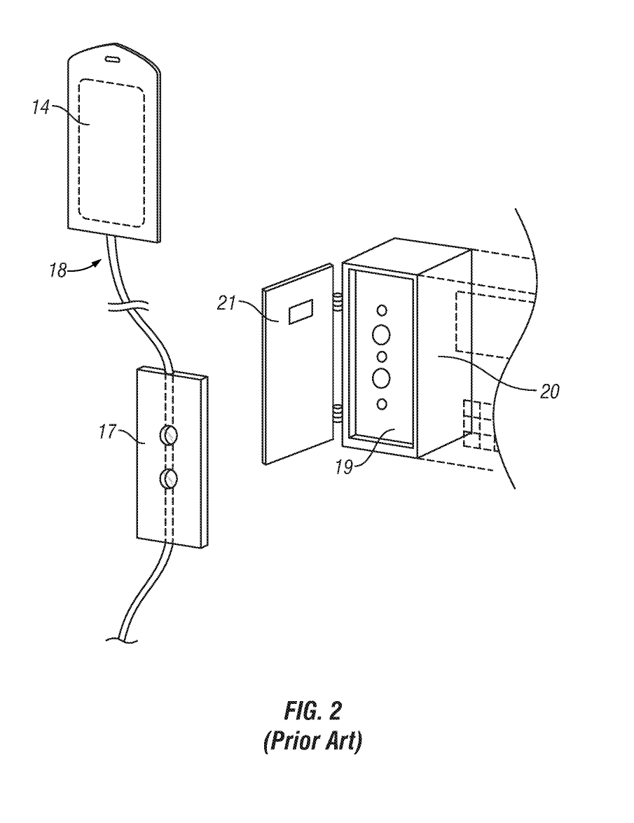

FIG. 2 illustrates a pumping segment 17 of a prior art IV set 18 fitting into the pump module of the IV pump of FIG. 1. In this example, pumping element 17 is integrally attached to the tubing of IV set 18 and includes two pumping chambers. The pumping module 20 of FIG. 1 is shown with its front door 21 opened and the pumping actuators 19 visible. When installed, the pumping element 17 is located against the pumping actuators 19 and door 21 closed to secure pumping element 17. Pumping element 17 will fill with fluid from fluid container 14 when valves or clamps (not shown) are opened. The pumping actuators 19 will then sequentially advance and compress portions of pumping element 17 to force fluid toward the patient 10 at a controlled flow rate.

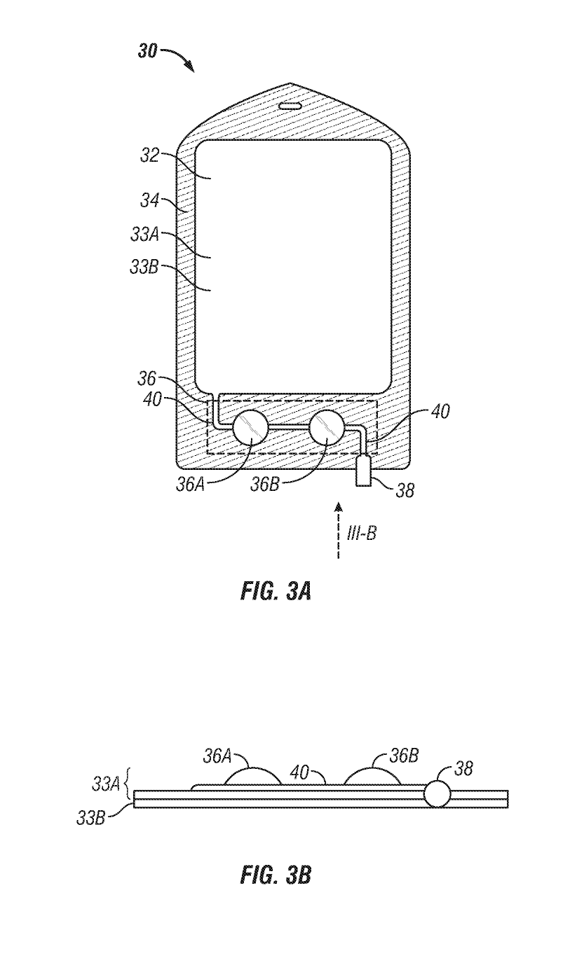

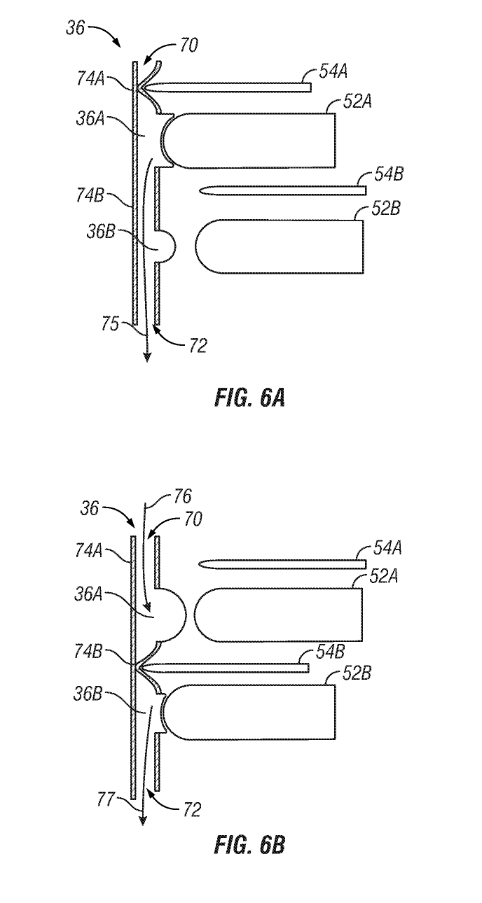

FIGS. 3A-3B respectively depict front and bottom views of a fluid container that includes a pumping element according to certain aspects of the present disclosure. In FIG. 3A, the fluid reservoir is formed from two flexible sheets 33A and 33B (overlaid in FIG. 3A and separately visible in FIG. 3B) that are sealed to each other in the region 34 that is indicated by hatching. This sealing may be accomplished by heat staking or other means of attachment such as bonding. As the two sheets 33A, 33B are not sealed in the area of reservoir 32, the space between the two sheets 33A, 33B forms the reservoir 32. Similarly, the two sheets 33A, 33B are not sealed to each other over the regions coincident with pumping chambers 36A, 36B and fluid conduits 40, thus enabling pumping chambers 36A, 36B and fluid conduits 40 to be formed at the same as the reservoir 32 is formed. In this embodiment, sheet 33A has been formed in the areas corresponding to pumping chamber 36A, 36B and over fluid conduits 40 while sheet 33B is flat. In other embodiments, both of sheets 33A, 33B are formed. The area indicated by the dashed-line box is the pumping element 36, including shut-off points that are depicted in FIGS. 6A-6B. An outlet 38 is attached to the bag 30. This connector outlet 38 may be the same type of connector as used for standard IV bags. In this example, the fluid conduits 40 are fluidically coupled between the reservoir 32 and the pumping chamber 36A, between pumping chambers 36A and 36B, and pumping chamber 36B and the outlet 38.

Compared to a standard IV bag, fluid container 30 provides an integral pumping element at a very small incremental cost. The amount of additional sheet material required to form the pumping element 36 is small, and may be zero with a redesign of the fluid container 30. Forming the pumping element 36 may be accomplished in the operation that forms the reservoir 32, and the net cost of a single tool to form both the reservoir 32 and the pumping element 36 may be less than the total cost of two separate tools.

FIG. 3B shows a bottom view of fluid container 30 in the direction of arrow III-B in FIG. 3A, wherein the pumping chambers 36A and 36B are visible as, in this example, deformable half domes. Fluid conduits 40 are also visible and are depicted as having a diameter thicker than the thickness of the two bonded sheets 33A, 33B in the illustrated embodiment. The outlet 38 is visible, in this example, as a circular connector suitable for a standard IV spike connection (not shown).

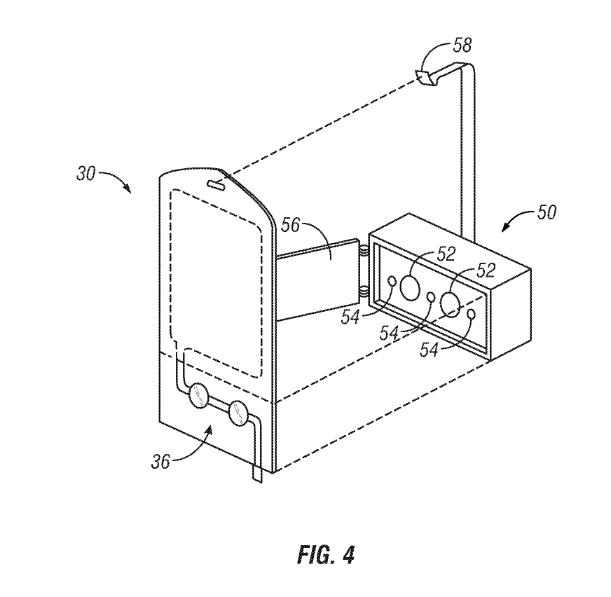

FIG. 4 depicts the fluid container 30 of FIG. 3A with an example IV pumping module 50 according to certain aspects of the present disclosure. This pumping module 50 may be used in place of the pumping module 20 of FIG. 1 and thus attached to control module 16. The portion of fluid container 30 that forms pumping module 36 is placed against the face of pumping module 50 and the door 56 closed. Pumping actuators 52 are configured to contact the pumping chambers 36A, 36B of pumping element 36 and occluders 52 are configured to stop the flow through fluid conduits 40 at various points as illustrated in FIGS. 6A and 6B. Fluid container 30 can be hung from a hook 58.

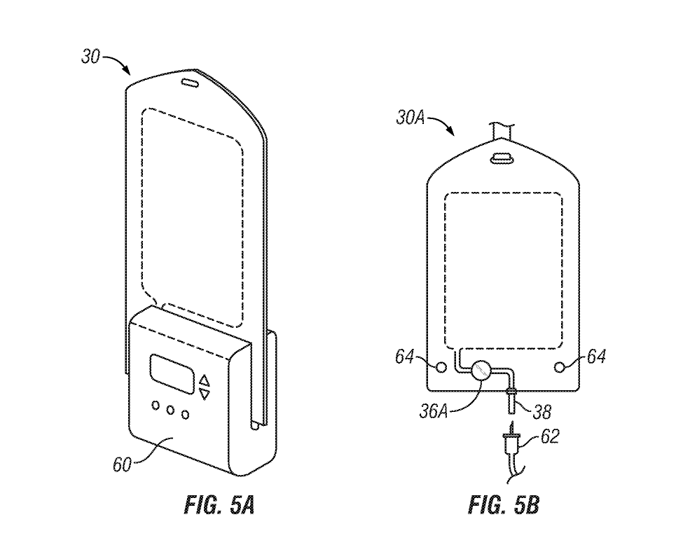

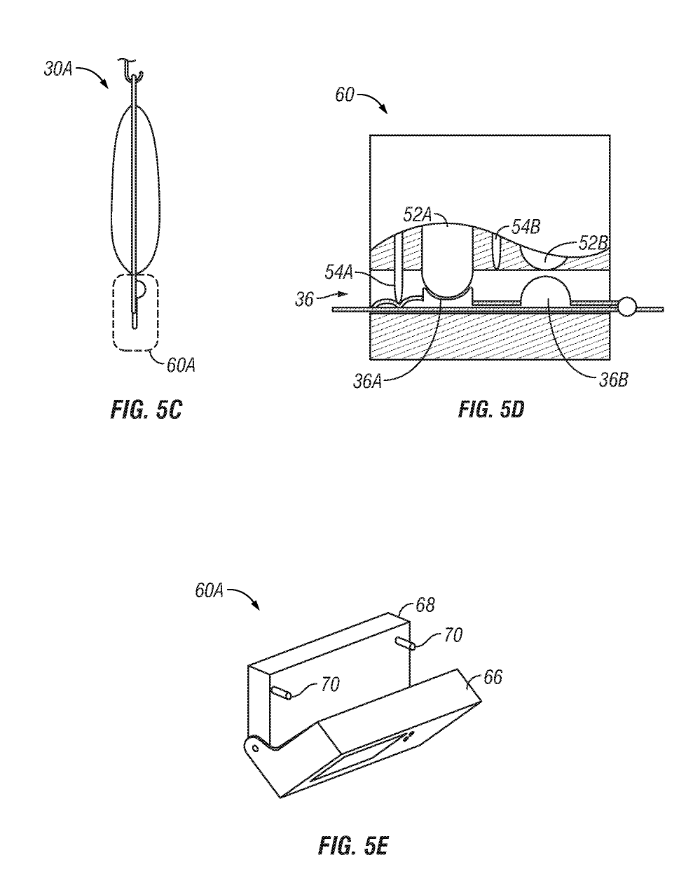

FIG. 5A depicts another embodiment of an IV pump 60 configured to operate in conjunction with the fluid container 30 of FIG. 3A according to certain aspects of the present disclosure. In this embodiment, the IV pump 60 may be a portable unit that may be battery powered. IV pump 60 clamps onto fluid container 30 using the attachment features 64 shown in FIG. 5B that are, in this example, holes in the sealed area of fluid container 30. Alternate attachment features may be pins, grommets, clips, or any other mechanism that would enable alignment and/or attachment of the IV pump 60 to the fluid container 30. The IV pump 60 of this example may be programmed manually or may have a wireless connection to another device to upload settings and download data.

FIGS. 5B-5C respectively depict front and side views of an embodiment of a fluid container 30A configured to operate with an attached pump according to certain aspects of the present disclosure. In this example, fluid container 30A has a single pumping chamber 36A and a connector 38 configured to accept a standard IV spike 62. In other embodiments, an IV set or a portion thereof may be integral to the fluid container 30A. Attachment features 64 provide a secure attachment for an IV pump such as IV pump 60. In FIG. 5C, the dashed line box 60A indicated the approximate volume where an IV pump may be attached.

FIG. 5D illustrates a portion of an example internal pumping mechanism of the pump 60 of FIG. 5A according to certain aspects of the present disclosure. In this example, pumping element 36 has been placed in the slot of IV pump 60 as shown in FIG. 5A. Pumping actuators 52A and 52B are aligned such that they compress pumping chambers 36A and 36B, respectively, when the pumping actuators 52A, 52B advance. In FIG. 5D, pumping actuator 52A is shown compressing pumping chamber 36B. Occluders 54A and 54B are configured to compress segments of fluid conduits 40 to prevent flow through those segments. In FIG. 5D, occluder 54A is shown compressing fluid conduit 40 to prevent flow. The operational sequence of occluders 54A, 54B and pumping actuators 52A, 52B is shown in FIGS. 6A and 6B.

FIG. 5E illustrates another embodiment of a pump 60A configured to operate in conjunction with the fluid container 30A of FIG. 5B according to certain aspects of the present disclosure. In this example, the primary electronics are contained in segment 66 that is hingedly or otherwise connected to a second segment 68. Pins 70 are configured to pass through alignment features 64 of fluid container 30A. To attach pump 60A to fluid container 30A, segments 66 and 68 are opened, as shown in FIG. 5E, and the pins 70 are inserted through alignment features 64 seen in FIG. 5B. Segments 66 and 68 are then closed, capturing fluid container 30A between them such that the pump 60A cannot separate from fluid container 30A. In other embodiments, other connecting arrangements are employed rather than the hinge configuration.

FIGS. 6A-6B illustrate an example operational sequence of the pumping element 36 of FIG. 5C. Similar operations are performed by the pumping module 50 of FIG. 4. Pumping element 36 is shown in cross-section and has two pumping chambers 36A and 36B. The occluders 54A and 54B and pumping actuator 52A and 52B of IV pump 50 are shown without the remaining structure for clarity. In the sections of fluid conduit 40 there are pinch points 74A and 74B identified where occluders 54A and 54B compress the fluid conduit 40 to stop flow. In this example, port 70 is the inlet, on the side of pumping element 36 connected to the fluid reservoir 32, and port 72 is on the side connected to the outlet 38. In FIG. 6A, occluder 54A is advanced, closing pinch point 74A and preventing flow out of port 70. Pumping actuator 52A is advancing and forcing fluid out port 72 as indicated by the arrow 75. Pumping actuator 52A will continue to advance at a rate selected to provide the desired rate of administration of the fluid to the patient 10 until the pumping chamber 36A has reached its minimum volume. At that time, occluder 54B advances, which closes pinch point 74B, and pumping actuator 52B begins to advance as shown in FIG. 6B. This expels fluid from pumping chamber 36B out port 72, as indicated by the arrow 77. At the same time, occluder 54A and pumping element 52A retract. As occluder 54B is preventing flow back from pumping chamber 36B, pumping chamber 36A refills from fluid container 32 (not shown) through port 70 as indicated by the arrow 76. This refill operation is completed before the volume of pumping chamber 36B is depleted.

When pumping chamber 36B reaches its minimum volume in the configuration of FIG. 6B, pumping chamber 36A has completed refilling. At that time, occluder 54A advances and occluder 54B retracts while pumping actuator 52A begins to advance while pumping actuator 52B begins to retract, thus returning the system to the configuration of FIG. 6A. Pumping chamber 36B is initially collapsed, however. It can be seen that, in this example, pumping chamber 36A is larger than pumping chamber 36B. As pumping actuator 52A begins to advance, some of the fluid expelled from pumping chamber 36A refills pumping chamber 36B. When pumping chamber 36B is fully expanded, then all of the fluid expelled from pumping chamber 36A exits port 72. In some configurations, pumping chamber 36A is twice the volume of pumping chamber 36B and the rate of advance of pumping actuator 52A is controlled such that the flow rate out of port 72 is approximately constant. In other configurations, the rate of flow may be variable over the pumping cycle.

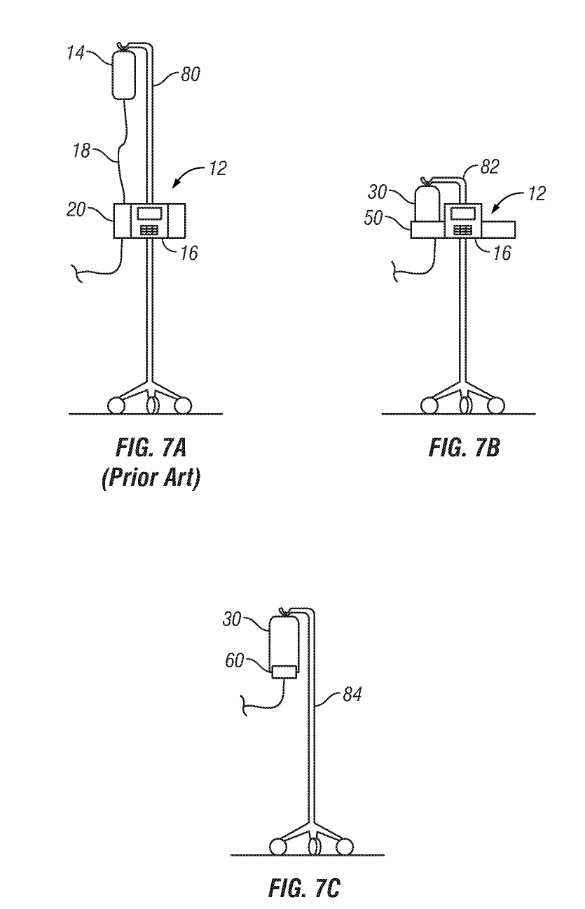

FIG. 7A depicts a prior art IV pump system. A typical system includes an IV pump 12, comprising control module 16 and pumping module 20, and a rolling IV pole 80 to which the IV pump 12 is mounted. IV pole 80 is typically 5-7 feet in height, with one or more hooks at the top to attach IV bags 14. With a filled IV bag 14 weighing 8 pounds or more at the top of the IV pole 80, the center of gravity (CG) is above the IV pump 12 and presents a risk to tip over while being moved around by the patient.

FIGS. 7B-7C depict IV pump systems incorporating various embodiments of fluid containers 30 and IV pumps 12 and 60 according to certain aspects of the present disclosure. FIG. 7B depicts an IV pump 12 where pumping module 50 has been substituted for pumping module 20 of FIG. 7A. As fluid container 30 hangs just above pumping module 50, the height of the IV pole 82 is less than that of IV pole 80 and thus the height of the CG of the IV pump system is reduced compared to that of FIG. 7A. FIG. 7C illustrates an IV pump 60 wherein the control module 16 and pump module 20 of FIG. 7A have been replaced by the clip-on IV pump 60. IV pole 84 is now a hanging point for fluid container 30, wherein the height is governed by the ease of use of a patient and the pumping pressure capabilities of IV pump 60. If desired, the fluid container 30 can be removed and carried by the patient rather than the patient having to roll the IV pole 84. While the CG of the IV pumping system of FIG. 7C is similar in height to that of FIG. 7B, the total weight may be lower and thus present less of a tipping hazard.

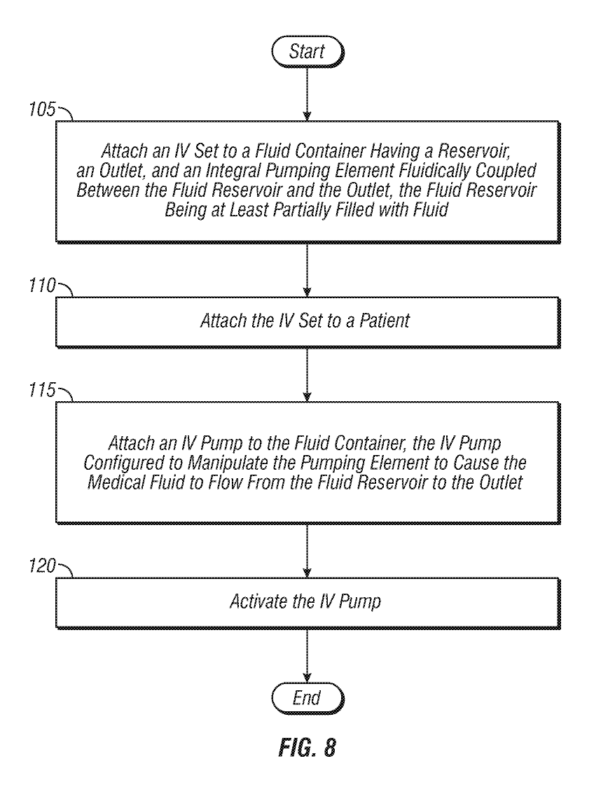

FIG. 8 is a flowchart illustrating the administration of a medical fluid using a fluid container 30 having an integral pumping element 36 according to certain aspects of the present disclosure. The system of FIGS. 3A and 5A will be used as an example system to describe this process. In step 105, the nurse attaches an IV set to a fluid container 30 having a fluid reservoir 32 and an outlet 38 with a pumping element 36 fluidically coupled between fluid reservoir 32 and outlet 38. The IV set may be connected to outlet 38. In step 110, the IV set is attached to a patient 10. In step 115, an IV pump, such as IV pump 60 of FIG. 5A, is attached to fluid container 30. In step 120, IV pump 60 is activated, causing fluid to flow from fluid reservoir 32 to patient 10.

It can be seen that the disclosed embodiments of the fluid container and IV pump provide a low-cost and convenient method of providing medical fluid to a patient. The integration of the pumping element into the fluid container increases the probability that the proper IV set is available at the proper location at the time of administration. The integrated fluid container also enables an elegant and smaller IV pumping system that is less prone to tip over in use and, in some embodiments, a portable system that can be hand-carried by the patient. The integration of the pumping element with the fluid container also reduces the risk of infection by providing a clean and sterile pumping element with every new fluid container rather than re-use of a pumping element in the IV set with the new IV bag.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the invention.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

1. An intravenous (IV) fluid container comprising:
a bag;
a fluid reservoir formed within the bag;
an outlet; and
a pumping element formed within the bag and fluidically coupled between the fluid reservoir and the outlet.

2. The IV fluid container of claim 1, wherein:
the bag comprises two flexible sheets that are sealed to each other in a sealing region; and
a first unsealed area of the two flexible sheets forms the fluid reservoir.

3. The IV fluid container of claim 2, wherein the pumping element comprises a pumping chamber formed by a second unsealed area of the two flexible sheets.

4. The IV fluid container of claim 3, wherein at least one of the flexible sheets is deformed over a portion of the pumping chamber.

5. The IV fluid container of claim 4, wherein the pumping element comprises at least two pumping chambers that are fluidically coupled between the fluid reservoir and the outlet.

6. The IV fluid container of claim 5, wherein the pumping chambers are fluidically coupled in series between the fluid reservoir and the outlet.

7. An IV pumping arrangement comprising:
a housing;
an attachment feature attached to the housing, the attachment feature configured to removably attach to an IV fluid container comprising:
a bag;
a fluid reservoir formed within the bag;
an outlet; and
a pumping element formed within the bag and fluidically coupled between the fluid reservoir and the outlet; and
an actuation feature attached to the housing, the actuation feature configured to manipulate the pumping element to cause fluid to flow from the fluid reservoir to the outlet.

8. The IV pumping arrangement of claim 7, wherein the pumping element comprises:
a pumping chamber;
an inlet fluid conduit fluidically coupled between the fluid reservoir and the pumping chamber; and
an outlet fluid conduit fluidically coupled between the pumping chamber and the outlet; and
wherein the actuation feature comprises:
a pumping actuator finger configured to selectively compress the pumping chamber; and
an occluder configured to selectively compress one of the fluid conduits of the pumping element.

9. The IV pumping arrangement of claim 7, wherein the pumping element comprises:
a fluid conduit fluidically coupled between the fluid reservoir and the outlet; and
wherein the actuation feature comprises:
a plurality of pumping actuator fingers configured to peristaltically compress the fluid conduit to cause fluid to flow from the fluid reservoir to the outlet.

10. The IV pumping arrangement of claim 7, wherein the IV fluid container further comprises an alignment feature formed in the body, and wherein the attachment feature comprises:
a clamping element movably attached to the body, the clamping element having a closed position wherein the clamping element grips the body of the IV fluid container;
an alignment element attached to either the body or the clamping element, the alignment element configured to mate with the alignment feature of the IV fluid container when the clamping element is in the closed position.

11. The IV pumping arrangement of claim 10, wherein:
the alignment feature comprises locator holes in the body of the IV fluid container;
the alignment element comprises pins that are configured to fit through the locator holes;
the pins are attached to one of the body or clamping element; and
the other of the body or clamping element is in contact with the pins when the clamping element is in the closed position such that the IV fluid container cannot be removed from the IV pump while the clamping element is in the closed position.

12. The IV pumping arrangement of claim 7, wherein the IV pump further comprises a power source.

13. The IV pumping arrangement of claim 12, wherein the IV pump is a portable device that attaches to the fluid container such that the fluid container and attached IV pump are a self-contained system that is separable from other structure or support.

14. An IV pumping system, comprising:
an IV fluid container comprising:
a body;
a fluid reservoir formed within the body;
a outlet attached to the body; and
a pumping element formed within the body and fluidically coupled between the fluid reservoir and the outlet; and
an IV pump configured to removably attach to the IV fluid container and manipulate the pumping element to cause fluid to flow from the fluid reservoir to the outlet.

15. The IV pumping system of claim 14, wherein the pumping element comprises:
a pumping chamber;
an inlet fluid conduit fluidically coupled between the fluid reservoir and the pumping chamber; and
an outlet fluid conduit fluidically coupled between the pumping chamber and the outlet.

16. The IV pumping system of claim 15, wherein the IV pump comprises:
a pumping actuator finger configured to selectively compress the pumping chamber; and
an occluder configured to selectively compress one of the fluid conduits of the pumping element.

17. The IV pumping system of claim 14, wherein the pumping element comprises:
a fluid conduit fluidically coupled between the fluid reservoir and the outlet; and
wherein the IV pump comprises:
a plurality of pumping actuator fingers configured to peristaltically compress the fluid conduit to cause fluid to flow from the fluid reservoir to the outlet.

18. The IV pumping system of claim 14, further comprising an IV pole to which the IV pump and the fluid container may be attached.

19. A method of providing a medical fluid to a patient, the method comprising the steps of:
attaching an IV set to the patient and to a fluid container having a reservoir, a outlet, and an integral pumping element fluidically coupled between the reservoir and the outlet, the reservoir being at least partially filled with the medical fluid;
attaching an IV pump to the fluid container, the IV pump configured to manipulate the pumping element to cause the medical fluid to flow from the reservoir to the outlet; and
activating the IV pump.

20. The method of claim 19, wherein the step of connecting the IV set comprises connecting the IV set to the outlet of the IV fluid container.
